(19) United States
(12) Patent Application Publication
(10) Pub. No.: US 2005/0123511 A1
(43) Pub. Date: Jun. 9, 2005

(54) Dna vaccine

(76) Inventors: David Thomas McCreavy, Liverpool (GB); William Duncan Fraser, Liverpool (GB); James Anthony Gallagher, Liverpool (GB)

Correspondence Address: CROWELL & MORING LLP;INTELLECTUAL PROPERTY GROUP, P.O. BOX 14300, WASHINGTON, DC, 20044-4300, US

(21) Appl. No.: 10/497,779

(22) PCT Filed: Dec. 6, 2002
(86) PCT No.: PCT/GB02/05512

(30) Foreign Application Priority Data
Dec. 7, 2001 (GB) 0129338.0
Oct. 12, 2002 (GB) 0223829.3

(51) Int. Cl. A61K 48/00; C12Q 1/70; C07H 21/04; C12N 15/86

(52) U.S. Cl. 424/93.2; 435/5; 530/388.1; 536/23.72; 435/456

(57) ABSTRACT

We describe vectors for use is DNA vaccination which are adapted such that nucleic acids which encode antigenic polypeptides are presented to the imune system in a folder or partially folded state to facilitate the production of antibodies to the native protein.

The invention relates to a DNA based vaccine for use in vaccinating animals, preferably humans, against disease and also for use in the generation of therapeutic antibodies and diagnostic antibodies; and including vectors adapted for DNA vaccination.

Antibodies developed through traditional techniques are used in a variety of both basic and clinical research applications including western blotting, immunoassay and immunohistochemistry. Essentially these reagents are derived from a two-step process of immune response induction and harvesting. During induction, a host, commonly a rat, mouse or rabbit is immunised of with progressively smaller quantities of immunogen over a predetermined time course. Initial exposure gives rise to a primary response in which low avidity IgM antibodies are the main neutralising species. A subset of B-cells termed memory cells are primed following this initial exposure, during subsequent encounters these trigger rapid clonal expansion of class switched IgG producing B-cells which play a role in rapidly neutralising the immunogen. A combination of somatic mutation and repeated exposure to the immunogen causes the host's immune system to preferentially clonally expand IgG producing B cells of highest affinity. During the harvesting, antibody is either collected in the form of polyclonal antisera or splenocytes are liberated and immortalised with a fusion partner to produce monoclonal antibodies.

A number of different forms of immunogen are used to induce an immune response. Commonly peptides based upon selected sequences, recombinant proteins and native purified proteins are used. To produce antibody reagents that bind with both high specificity and high affinity (monoclonal), avidity (polyclonal) and are therefore of utility in immunoassay, induction of an authentic native response (ANR) is vital. ANR occurs when the immunogen presented to the host is folded correctly ie of the correct conformation and is also post-translationally modified in a tissue specific manner. The interaction of antibody with antigen is based upon complimentarily. The antigen must provide a 3D surface with a sufficiently distinct contour (epitope) to enable an antibody with a reciprocal contour to bind to. During immunisation with peptides good binding may be observed however the utility of the antibody is determined by how well the peptide mimics the epitope of the native molecule. Generally as the peptide lacks the structure of the native molecule its conformation is far removed and therefore its ability to mimic is limited. Immunisation with recombinant proteins addresses this issue by producing large regions of the protein with numerous epitopes, these are potentially able to mimic the native molecule. However recombinant proteins do not undergo post-translational modifications (FTM). These are a range of alterations that occur in mammalian cells that can change the form of the molecule profoundly, resulting in the production of new epitopes and the masking of existing ones. Inmunisation with native proteins potentially addresses both issues, however in many cases it is practically impossible to isolate and purify sufficient protein and were this does occur the integrity of the isolate may be questioned due to detrimental effects the techniques employed.

The challenges are not limited to the source of immunogen, the region of the molecule used for immunisation may have an important effect on the utility of the reagents produced. By way of example it may be known at the messenger level that breast tumour cells overexpress protein X which encodes for a 200 amino acid protein and may therefore potentially be a marker of the tumour. However the tumour cells may also over express a number of prohormone convertases which act on substrates within the Protein X primary sequence resulting in the secretion of protein X1-50 only. Unless the secreted form is known in advance the production of antibodies using the aforementioned immunogens becomes somewhat more demanding.

One of the most important developments in recent medical history is the development of vaccines which provide prophylactic protection from a wide variety of pathogenic organisms. Many vaccines are produced by inactivated or attenuated pathogens which are injected into an individual. The immunised individual responds by producing both a humoral (antibody) and cellular (cytolytic T cells, CTL's) responses. For example, hepatitis vaccines are made by heat inactivating the virus and treating it with a cross linking agent such as formaldehyde. An example of an attenuated pathogen useful as a vaccine is represented by polio vaccines which are produced by attenuating a live pathogen.

However the use of attenuated organisms in vaccines for certain diseases is problematic due to the lack of knowledge regarding the pathology of the condition and the nature of the attenuation. For certain viral agents this is a particular problem since viruses, in particular retroviruses, have an error prone replication cycle which results viable mutations in the genes which comprise the virus. This can result in alterations to antigenic determinants which have previously been used as vaccines. An alternative to the use of inactivated or attenuated pathogens is the identification of pathogen epitopes to which the immune system is particularly sensitive. In this regard many pathogenic toxins produced by pathogenic organisms during an infection are particularly useful in the development of vaccines which protect the individual from a particular pathogenic organism.

The development of so-called subunit vaccines (vaccines in which the immunogen is a fragment or subunit of a protein or complex expressed by a particular pathogenic organism) has been the focus of considerable medical research. The need to identify candidate molecules useful in the development of subunit vaccines is apparent not least because conventional chemotherapeutic approaches to the control of pathogenic organisms has more recently been stymied by the development of antibiotic resistance.

It has recently been observed that the technique of DNA vaccination can produce antibodies. This technique involves transfecting cells in vivo with a plasmid vector containing a gene encoding the protein immunogen. During transfection the plasmid enters the cell and resides within the cytoplasm where transcription and translation occurs. The protein is subsequently digested by the proteasome (a multi-subunit protease found in the cytoplasm of eukaryotes and some bacteria and archeabacteria) and the digested fragments transferred to the endoplasmic reticulum where they become bound to MHC class I proteins, which become displayed on the cell surface triggering the cell-mediated response mechanisms. As all cells express MHC class I the outcome of injection of plasmid DNA is heavily biased towards a cell mediated effect in which the injection of the plasmid DNA is akin to viral infection, the production of antibodies appears to be a minor effect in these vectors. However potentially DNA vaccination offers a number of features which overcome the limitations of eliciting an immune response through the aforementioned traditional routes. Namely (i) if the gene sequence encoding the immunogen of interest is known all potential epitopes will be expressed, (ii) within the limitations of host-human gene conservation the translated gene will be correctly post-translationally modified, cleaved, packaged and secreted. (iii) Integral proteins such as transmembrane receptors will be incorporated within the cell membrane presenting correctly folded extracellular domains to the immune system.

As the vector produces the protein in an endogenous manner these are perfectly placed to induce HLA I responses giving rise to CD8+ cytotoxic effects. All nucleated cells display HLA I, therefore the cells that the plasmid transfects will process the expressed protein, by degrading into peptides of 7-13 amino acids via the proteasome, transporting these by a heterodimeric peptide transporter associated with antigen processing (TAP) 1 and 2 molecules into the endoplasmic reticulum where the resultant peptides are bound to HLA I and 2 microglobulin prior to being displayed on the surface of the cell.

The same effects are believed to occur when mammalian cells are transfected in vitro. Therefore, the production of antibodies within this framework occurs as an aside, when the cell is lysed during the CD8+ response and some of the expressed protein, which has not been degraded by the proteasome, is released into the extracellular environment where it induces a HLA II response from antigen presenting and B-cells.

The in vivo transfection of DNA offers the potential to elicit immune responses to expressed protein which will significantly surpass any of the traditional methods for raising antibodies. The reason for this is that 50-90% of all proteins natively undergo post-translational modification which gives rise to natural epitopes. Consequently utilising the hosts own cellular machinery will enable the antigen to be presented to the immune system in its native state, (ie post-translationally modified) thereby inducing the most suitable binding via B-cell receptors and hence antibody production. To achieve this transgenic mRNA may be preserved by inhibiting post-transcriptional gene silencing (PTGS) and the activity of the proteasome inhibited in order to allow the expressed protein (antigen) to be secreted or integrated into the cell membrane to facilitate a HLA II antibody effect.

Posttranscriptional gene silencing (PTGS) is a recently discovered phenomenon in which sequence specific mRNA degradation occurs following the introduction of transgenes into cells (Cogino et al., 2000). Small interfering RNA's of 21-25 nucleotides are generated from larger RNA strands, once produced these anneal to mRNA transcripts and target them for degradation by an as yet uncharacterized enzyme complex. A candidate molecule for both the cleavage of the siRNA precursor and the enzyme complex has been identified and is termed Dicer in Drosophila. (Moss 2001). Several studies in humans, flies and worms have demonstrated that Dicer (or species homolog) expression is required to produce siRNA's and facilitate PTGS (Grishok et al., 2001, Hutvagner et al., 2001, Knight et al., 2001). Down regulation of the recently cloned mammalian homolog A HERNA (helicase-MOI) accession number AB028449 (Matsuda et al., 2000) through its antisense incorporation into mammalian expression vectors offer the potential to increase the transcriptional efficiency of said vectors resulting in greater levels of transgene expression.

There are a number of proteins known to act as proteosome inhibitors. For example, Etlinger et al have identified two proteins which inhibit the activity of the proteosome. These proteins have molecular weights of 240,000 and 200,000 Daltons which are homomultimers of a 40,000 and 50,000 Dalton subunits. In addition, P131 (Li et al 1992) is believed to be an effective proteasomal inhibitor. The proteosome is a multi-subunit protease consisting of 28 subunits arranged in 4 heptameric rings stacked upon one another to form a cylinder shaped particle of 700,000 Daltons. McCutcheon-Maloney et al. (Journal of Biol. Chem 275 (24):18557) discloses the nucleic acid sequence of human P131 which has a molecular weight of 29.8 kDa.

Alternatively expression of protein subunits of the proteasome could be inhibited thought the incorporation within the vector of antisense nuclei acid sequences or inhibitory RNA molecules. Specifically the following sequences could be targeted, HC2 (accession no. D00759), HC3 (accession no.D00760), HC8 (accession no. D00762), HC9 (accession no. D00763), macropain zeta (accession no.X61970), PROS.27 (accession no.X59417) and XAPC7 (accession no. AF022815).

The invention relates to the provision of a vector which includes an antigenic, preferably a CD4⁺, T cell specific heterologous nucleic acid molecule encoding an antigenic polypeptide which further includes a nucleic acid molecule which encodes a protease inhibitor, typically an inhibitor of the proteosome protease.

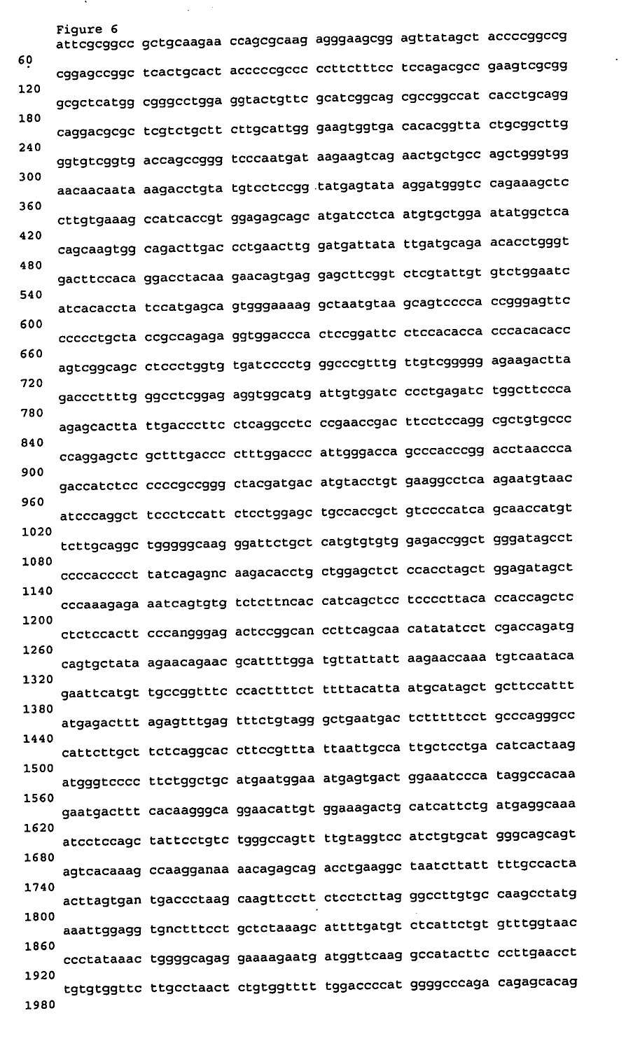

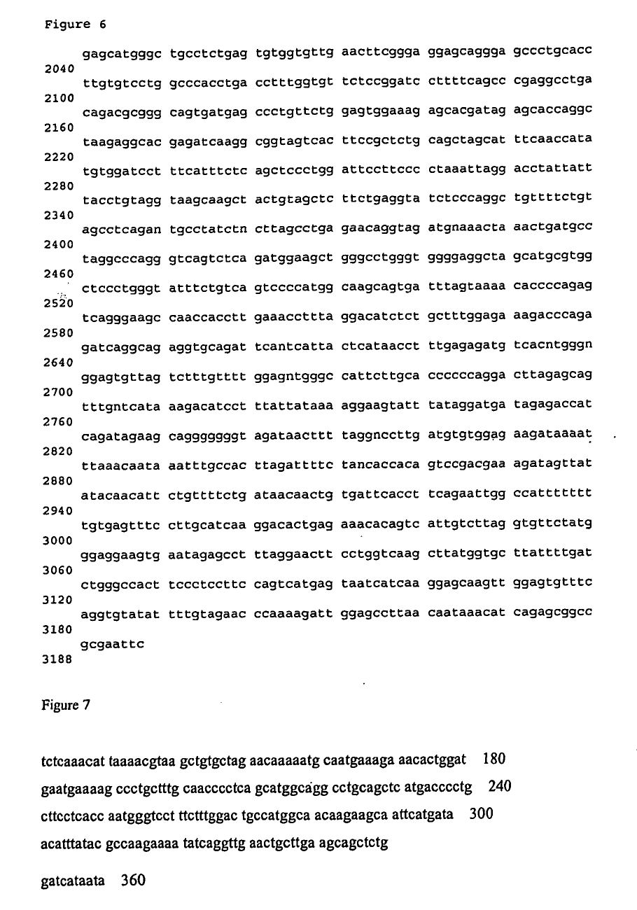

According to a first aspect of the invention there is provided a vector comprising a heterologous nucleic acid sequence encoding an antigenic polypeptide and a further nucleic acid molecule selected from the group consisting of;
i) a nucleic acid molecule comprising a nucleicacid sequence as represented in FIG. 6;
ii) a nucleic acid molecule which hybridizes to the nucleic acid molecule in FIG. 6 and which encodes a protease inhibitor polypeptide;
iii) a nucleic acid molecules which comprise nucleic acid sequences which are degenerate because of the genetic code to the sequences in (i) and (ii) above.
   wherein said vector is adapted for the expression of each polypeptide.

In a preferred embodiment of the invention said vector is selected from the group consisting of: a plasmid; a phagemid, a virus.

In further preferred embodiment of the invention said viral based vector is based on viruses selected from the group consisting of: adenovirus; retrovirus; adeno associated virus; herpesvirus; lentivirus; baculovirus.

In a further preferred embodiment of the invention said heterologous nucleic acid sequence encodes an antigenic polypeptide derived from a viral pathogen.

In a yet further preferred embodiment of the invention said viral pathogen is selected from the group consisting of: Human Immunodeficiency Virus (HIV1 & 2 e.g. gp120 portion of the HIV-1 envelope protein); Human T Cell Leukamia Virus (HTLV 1 & 2); Ebola virus; human papilloma virus(HPV); papovavirus; rhinovirus; poliovirus; herpesvirus; adenovirus; Epstein barr virus; influenza virus.

In a further preferred embodiment of the invention said heterologous nucleic acid sequence encodes an antigenic polypeptide derived from a bacterial pathogen.

In a yet further preferred embodiment of the invention said bacterial pathogen is selected from the group consisting of: Staphylococcus aureus; Staphylococcus epidermidis; Enterococcus faecalis; Mycobacterium tuberculsis; Streptococcus group B; Streptoccocus pneumoniae; Helicobacter pylori (e.g. the VacA and CagA proteins); Neisseria gonorrhea; Streptococcus group A; Borrelia burgdorferi; Coccidiodes immitis; Histoplasma sapsulatum; Neisseria meningitidis type B; Shigella flexneri; Escherichia coli; Haemophilus influenzae.

In a further preferred embodiment of the invention said heterologous nucleic acid sequence encodes an antigenic polypeptide derived from a parasitic pathogen, e.g. Wb-SXP-1, and BM-SXP-1 proteins of Brugian and Bancroftian filariasis.

In a yet further preferred embodiment of the invention said parasitic pathogen is selected from the group consisting of: Trypanosoma Brucei spp (e.g. p67 protein).; Plasmodiurn spp.

In a further preferred embodiment of the invention said heterologous nucleic acid sequence encodes an antigenic polypeptide derived from a fungal pathogen.

In a yet further preferred embodiment of the invention said fungal pathogen is Candida spp, preferably Candida albicans (e.g. hsp90 protein)

In a further preferred embodiment of the invention said heterologous nucleic acid sequence encodes an antigen which is tumour specific. Preferably said tumour specific antigen is selected from the group consisting of: MAGE, BAGE, GAGE and DAGE families of tumour rejection antigen precursor. A further example of a tumour specific antigen is parathyroid hormone related protein, cathepsin K (both in breast cancer), prostate specific antigen in prostate cancer.

Tumour rejection antigens are well known in the art and include, by example and not by way of limitation, the MAGE, BAGE, GAGE and DAGE families of tumour rejection antigens, see Schulz et al Proc Natl Acad Sci USA, 1991, 88, pp991-993.

It will be apparent to one skilled in the art that the vector according to the invention could comprise a heterologous nucleic acid which encodes a polypeptide associated with a pathological condition to immunise an animal against a selected polypeptide to provide either prophylatic protection or to provide a therapy against disease provoking agents (eg viruses, bacteria) or diseases such as cancer. Alternatively the vector according the invention could be used to generate antibodies to polypeptides which have utility either as therapeutic antibodies or as diagnostic antibodies.

In a further preferred embodiment of the invention said vector is an expression vector adapted for expression in a eukaryotic cell.

As used herein, a “vector” may be any of a number of nucleic acids into which a desired sequence may be inserted. Vectors include, but are not limited to, plasmids, phagemids and virus genomes. A cloning vector is one which is able to replicate in a host cell, and which typically is further characterized by one or more endonuclease restriction sites at which the vector may be cut in a determinable fashion and into which a desired DNA sequence may be ligated such that the recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

Vectors may further contain one or more selectable marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase), and genes which visibly affect the phenotype of transformed or transfected cells, hosts, colonies or plaques (e.g., various fluorescent proteins such as green fluorescent protein, GFP). Preferred vectors are those capable of autonomous replication, also referred to as episomal vectors. Alternatively vectors may be adapted to insert into a chromosome, so called integrating vectors. The vector of the invention is typically provided with transcription control sequences (promoter sequences) which mediate cell/tissue specific expression. These promoter sequences may be cell/tissue specific, inducible or constitutive.

Promoter is an art recognised term and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences and is therefore position independent). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of environmental cues which include, by example and not by way of limitation, intermediary metabolites (eg glucose, lipids), environmental effectors (eg heat).

Promoter elements also include so called TATA box, RNA polymerase initiation selection (RIS) sequences and CAAT box sequence elements which function to select a site of transcription initiation. These sequences also bind polypeptides which function, inter alia, to facilitate transcription initiation selection by RNA polymerase.

Adaptations also include the provision of autonomous replication sequences which both facilitate the maintenance of said vector in either the eukaryotic cell or prokaryotic host, so called “shuttle vectors”. Vectors which are maintained autonomously are referred to as episomal vectors. Episomal vectors are desirable since these molecules can incorporate large DNA fragments (30-50 kb DNA). Episomal vectors of this type are described in WO98/07876.

Adaptations which facilitate the expression of vector encoded genes include the provision of transcription termination/polyadenylation sequences. This also includes the provision of internal ribosome entry sites (IRES) which function to maximise expression of vector encoded genes arranged in bicistronic or multi-cistronic expression cassettes.

Expression control sequences also include so-called Locus Control Regions (LCRs). These are regulatory elements which confer position-independent, copy number-dependent expression to linked genes when assayed as transgenic constructs in mice.

LCRs include regulatory elements that insulate transgenes from the silencing effects of adjacent heterochromatin, Grosveld et al., Cell (1987), 51: 975-985.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc.(1994).

In a preferred embodiment of the invention the expression of said heterologous nucleic acid molecule is controlled by its cognate promoter. A “cognate promoter” is a promoter which would naturally control the expression of the gene from which said heterologous nucleic acid was derived. For example, and not by way of limitation, the use of a HIV long terminal repeat to control the expression of an HIV encoded polypeptide.

Alternatively, said heterologous nucleic acid is controlled by a promoter which does not naturally control the expression of the gene from which said heterologous nucleic acid was derived. For example, and not by way of limitation, the use of a muscle specific promoter (e.g Myo D) to regulate expression of an HIV encoded polypeptide.

In a preferred embodiment of the invention said promoter is a constitutive promoter. Preferably said promoter is selected from the group consisting of: CMV; SV40; chicken beta actin; CMVie enhanced; telomerase reverse transcriptase; H⁺/K⁺ ATPase; glyceraldehyde-3-phosphate dehydrogenase (GAPDH).

In a further preferred embodiment of the invention said promoter is a regulatable promoter, preferably a cell or tissue specific promoter. Preferably said tissue specific promoter is selected from the group consisting of: alkaline phosphatase; albumin; casein; prostate specific antigen; osteocalcin; cathepsin K; TRAP; RankL; PC8; cytokeratins 1,6,9,10,14,16; collagen type 1; elastin; NF-ATI (NF-Atp, NF-Atc2); tyrosinase; TRP-1, and muscle specific creatine kinase.

More preferably still said promoter is a muscle specific promoter, for example, MCK or myosin light chain 3F.

Muscle specific promoters are known in the art. For example, WO0009689 discloses a straited muscle preferentially expressed gene and cognate promoter, the SPEG gene. EP1072680 discloses the regulatory region of the myostatin gene. The gene shows a predominantly muscle specfic pattern of gene expression. US5795872 discloses the use of the creatine kinase promoter to achieve high levels of expression of foreign proteins in muscle tissue. The muscle specific gene Myo D also shows a pattern of expression restricted to myoblasts.

In a further preferred embodiment of the invention said protease inhibitor is an inhibitor of the proteosome.

In a yet further preferred embodiment of the invention said protease inhibitor is mammalian PI31, preferably human PI31.

In a further preferred embodiment of the invention expression of the PI31 nucleic acid is controlled by its cognate promoter. Alternatively said PI31 gene is controlled by a promoter which does not naturally control expression of the PI31 gene.

In a further preferred embodiment of the invention the PI31 nucleic acid is expressed co-ordinantly with said heterologous nucleic acid.

It will be apparent to one skilled in the art that co-ordinant expression may be achieved in several ways. For example placing both nucleic acids under the control of the same promoter. This can be achieved by, either constructing an expression cassette which places the heterologous nucleic acid and the PI31 gene under the control of a single promoter. Alternatively the heterologous nucleic acid can be placed under the control of separate promoters which are expressed co-ordinantly.

In a further preferred embodiment of the invention said vector is provided a nucleic acid molecule which encodes a polypeptide which stimulates the expression of MHC class II.

In a preferred embodiment of the invention said nucleic acid molecule is selected from the group consisting of:
i) a nucleic acid molecule comprising a nucleicacid sequence as represented in FIG. 5;
ii) a nucleic acid molecule which hybridizes to the nucleic acid molecule in FIG. 5 and which encodes a polypeptide which stimulates MHC class II expression;
iii) a nucleic acid molecules which comprise nucleic acid sequences which are degenerate because of the genetic code to the sequences in (i) and (ii) above.

In a preferred embodiment of the invention said polypeptide is selected from the group consisting of: RFX5, RFXAP, CIITA, or sequence homologue thereof.

Preferably said polypeptide is CIITA (DNA accession number U60653).

In a further preferred embodiment of the invention said vector is yet further adapted to express an inhibitory RNA molecule wherein said inhibitory RNA is expressed from a DNA molecule selected from the group consisting of:
i) a DNA molecule comprising a DNA sequence as represented in FIG. 7;
ii) a DNA molecule which hybridizes to the sequence in FIG. 7 and which has helicase activity;
iii) a DNA molecule which is degenerate because of the genetic code to those sequences in (i) and (ii) above.

A number of techniques have been developed in recent years which purport to specifically ablate genes and/or gene products. A recent technique to specifically ablate gene function is through the introduction of double stranded RNA, also referred to as inhibitory RNA (RNAi), into a cell which results in the destruction of mRNA complementary to the sequence included in the RNAi molecule. The RNAi molecule comprises two complementary strands of RNA (a sense strand and an antisense strand) annealed to each other to form a double stranded RNA molecule. The RNAi molecule is typically derived from exonic or coding sequence of the gene which is to be ablated. Surprisingly, only a few molecules of RNAi are required to block gene expression which implies the mechanism is catalytic. The site of action appears to be nuclear as little if any RNAi is detectable in the cytoplasm of cells indicating that RNAi exerts its effect during mRNA synthesis or processing.

An alternative embodiment of RNAi involves the synthesis of so called stem loop RNAi molecules which are synthesised from expression cassettes carried in vectors. The DNA molecule encoding the stem-loop RNA is constructed in two parts, a first part which is derived from a gene the regulation of which is desired. The second part is provided with a DNA sequence which is complementary to the sequence of the first part. The cassette is typically under the control of a promoter which transcribes the DNA into RNA. The complementary nature of the first and second parts of the RNA molecule results in base pairing over at least part of the length of the RNA molecule to form a double stranded hairpin RNA structure or stem-loop. The first and second parts can be provided with a linker sequence. Stem loop RNAi has been successfully used in plants to ablate specific mRNA's and thereby affect the phenotype of the plant, see Smith et al (2000) Nature 407, 319-320. Typically, RNAi molecules of less than 50 nucleotides are effective although longer double stranded molecules have efficacy. Molecules of approximately 20 nucleotides work particularly well.

According to a further aspect of the invention there is provided a method to induce an immune response to an antigenic polypeptide comprising administering to an animal, preferably a human, the vector according to any previous aspect or embodiment.

In a preferred method of the invention said vector is, for example, administered by oral, intravenous, intraperitoneal, intramuscular, intracavity, subcutaneous, or transdermal injection.

According to a further aspect of the invention there is provided an antibody obtainable by the method according to the invention.

In a preferred embodiment of the invention said antibody is a therapeutic antibody.

In an further preferred embodiment of the invention said antibody is a diagnostic antibody. Preferably said diagnostic antibody is provided with a label or tag.

In a preferred embodiment of the invention said antibody is a monoclonal antibody or active binding fragment thereof. Preferably said antibody is a humanised or chimeric antibody.

A chimeric antibody is produced by recombinant methods to contain the variable region of an antibody with an invariant or constant region of a human antibody.

A humanised antibody is produced by recombinant methods to combine the complimentarity determining regions of an antibody with both the constant (C) regions and the framework regions from the variable (V) regions of a human antibody.

Antibodies, also known as immunoglobulins, are protein molecules which have specificity for foreign molecules (antigens). Immunoglobulins (Ig) are a class of structurally related proteins consisting of two pairs of polypeptide chains, one pair of light (L) (low molecular weight) chain (κ or λ), and one pair of heavy (H) chains (γ, α, μ, δ and ε), all four linked together by disulphide bonds. Both H and L chains have regions that contribute to the binding of antigen and that are highly variable from one Ig molecule to another. In addition, H and L chains contain regions that are non-variable or constant.

The L chains consist of two domains. The carboxy-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region.

The H chains of Ig molecules are of several classes, α, μ, σ, α, and γ (of which there are several sub-classes). An assembled Ig molecule consisting of one or more units of two identical H and L chains, derives its name from the H chain that it possesses. Thus, there are five Ig isotypes: IgA, IgM, IgD, IgE and IgG (with four sub-classes based on the differences in the H chains, i.e., IgG1, IgG2, IgG3 and IgG4). Further detail regarding antibody structure and their various functions can be found in, Using Antibodies: A laboratory manual, Cold Spring Harbour Laboratory Press.

Chimeric antibodies are recombinant antibodies in which all of the V-regions of a mouse or rat antibody are combined with human antibody C-regions. Humanised antibodies are recombinant hybrid antibodies which fuse the complimentarity determining regions from a rodent antibody V-region with the framework regions from the human antibody V-regions. The C-regions from the human antibody are also used. The complimentarity determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not illicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

In a further preferred embodiment of the invention said antibodies are opsonic antibodies.

Phagocytosis is mediated by macrophages and polymorphic leukocytes and involves the ingestion and digestion of micro-organisms, damaged or dead cells, cell debris, insoluble particles and activated clotting factors. Opsonins are agents which facilitate the phagocytosis of the above foreign bodies. Opsonic antibodies are therefore antibodies which provide the same function. Examples of opsonins are the Fc portion of an antibody or compliment C3.

In another aspect of the invention there is provided a vector which is adapted for the expression of the humanised or chimeric antibodies according to the invention.

In a yet further aspect of the invention, there is provided a cell or cell line which has been transformed or transfected with the vector encoding the humanised or chimeric antibody according to the invention.

In a yet further aspect of the invention there is provided a method for the production of the humanised or chimeric antibody according to the invention comprising:
(i) providing a cell transformed or transfected with a vector which comprises a nucleic acid molecule encoding the humanised or chimeric antibody according to the invention;
(ii) growing said cell in conditions conducive to the manufacture of said antibody; and
(iii) purifying said antibody from said cell, or its growth environment.

In a yet further aspect of the invention there is provided a hybridoma cell line which produces a monoclonal antibody as hereinbefore described.

In a further aspect of the invention there is provided a method of producing monoclonal antibodies according to the invention using hybridoma cell lines according to the invention.

In a further aspect of the invention there is provided a method for preparing a hybridoma cell-line producing monoclonal antibodies according to the invention comprising the steps of:
i) immunising an immunocompetent mammal with the vector according to the invention;
ii) fusing lymphocytes of the immunised immunocompetent mammal with myeloma cells to form hybridoma cells;
iii) screening monoclonal antibodies produced by the hybridoma cells of step (ii) for binding activity to the amino acid sequence encoded by the heterologous nucleic acid according to the invention;
iv) culturing the hybridoma cells to proliferate and/or to secrete said monoclonal antibody; and
v) recovering the monoclonal antibody from the culture supernatant.

Preferably, the said immunocompetent mammal is a mouse. Alternatively, said immunocompetent mammal is a rat.

According to a yet further aspect of the invention there is provided a vaccine comprising the vector according to the invention.

In a preferred embodiment of the invention said vaccine further includes an adjuvant.

An adjuvant is a substance or procedure which augments specific immune responses to antigens by modulating the activity of immune cells. Examples of adjuvants include, by example only, agonsitic antibodies to co-stimulatory molecules, Freunds adjuvant, muramyl dipeptides, liposomes. An adjuvant is therefore an immunomodulator. It is envisaged that an adjuvant may be administered simultaneously, sequentially or separately with the vector according to the invention thereby augmenting an immune response to the polypeptide encoded by the heterologous nucleic acid.

Liposomes, as well as having an adjuvant effect may also serve as a carrier for the vector according to the invention. Liposomes are lipid based vesicles which encapsulate a selected therapeutic agent (ie a vector) which is then introduced into a patient. The liposome is manufactured either from pure phospholipid or a mixture of phospholipid and phosphoglyceride. Typically liposomes can be manufactured with diameters of less than 200 nm, this enables them to be intravenously injected and able to pass through the pulmonary capillary bed. Furthermore the biochemical nature of liposomes confers permeability across blood vessel membranes to gain access to selected tissues.

Liposomes do have a relatively short half-life. So called STEALTH® liposomes have been developed which comprise liposomes coated in polyethylene glycol (PEG). The PEG treated liposomes have a significantly increased half-life when administered intravenously to a patient. In addition STEALTH® liposomes show reduced uptake in the reticuloendothelial system and enhanced accumulation selected tissues. In addition, so called immuno-liposomes have been develop which combine lipid based vesicles with an antibody or antibodies, to increase the specificity of the delivery of the vector to a selected cell/tissue.

The use of liposomes as delivery means is described in U.S. Pat. Nos. 5,580,575 and 5,542,935.

In a further aspect of the invention there is provided a method to vaccinate an animal, preferably a human, against at least one pathological condition.

In a preferred method of the invention said pathological condition is an infection caused by a virus. Preferably said viral infection is selected from the group consisting of: AIDS; herpes; rubeola; rubella; varicella; influenza; common cold; viral meningitis.

In a further preferred method of the invention said pathological condition is an infection caused by a bacterium. Preferably said bacterial infection is selected from the group consisting of: septicaemia; tuberculosis; bacteria-associated food poisoning; blood infections; peritonitis; endocarditis; sepsis; bacterial meningitis; pneumonia; stomach ulcers; gonorrhoea; strep throat; streptococcal-associated toxic shock; necrotizing fasciitis; impetigo; histoplasmosis; Lyme disease; gastro-enteritis; dysentery; shigellosis.

In a further preferred method of the invention said pathological condition is an fungal infection. Preferably said fungal infection is candidiasis.

In a further preferred method of the invention said pathological condition is a parasitic infection. Preferably said parasitic infection is selected from the group consisting of: trypanosomiasis; malaria; schistosomiasis; Chagas disease.

In addition to the development of vaccines to combat diseases, DNA vaccination using the vector according to the invention, will have utility with respect to the development of diagnostic agents. For example for use in detecting the expression of polypeptide markers in biological samples or to monitor environmental agents in, for example soil samples. Non pathological polypeptides may also be monitored for example, in plants the VAP27-1 and VAP27-2 proteins.

An embodiment of the invention will now be described by example only and with reference to the following table and figures:

Table 1 is a summary of vector construct features;

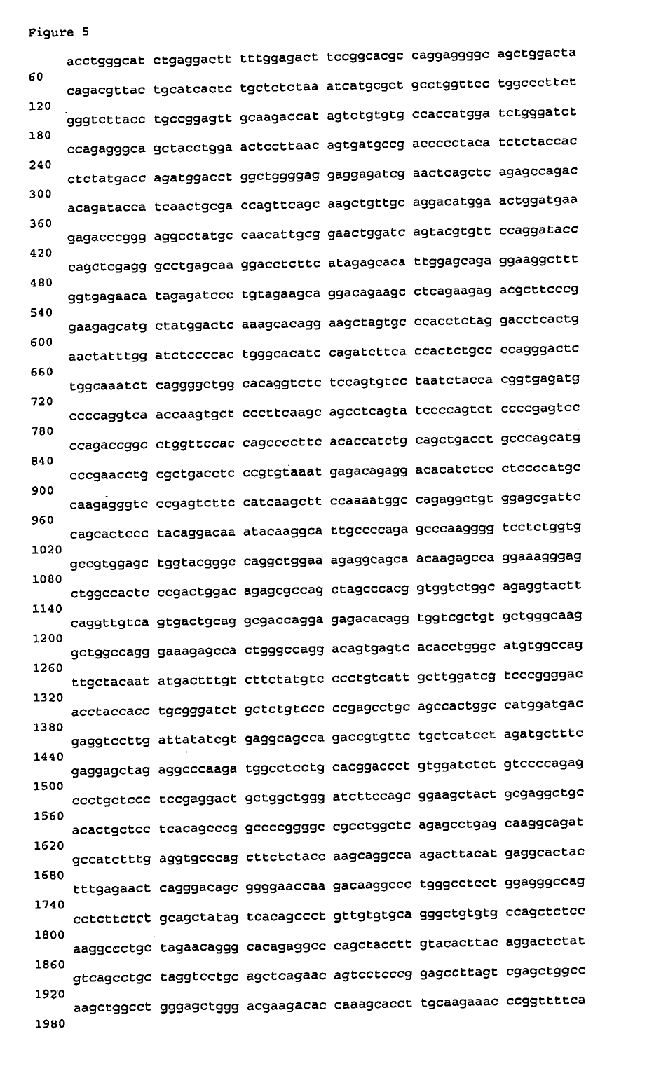

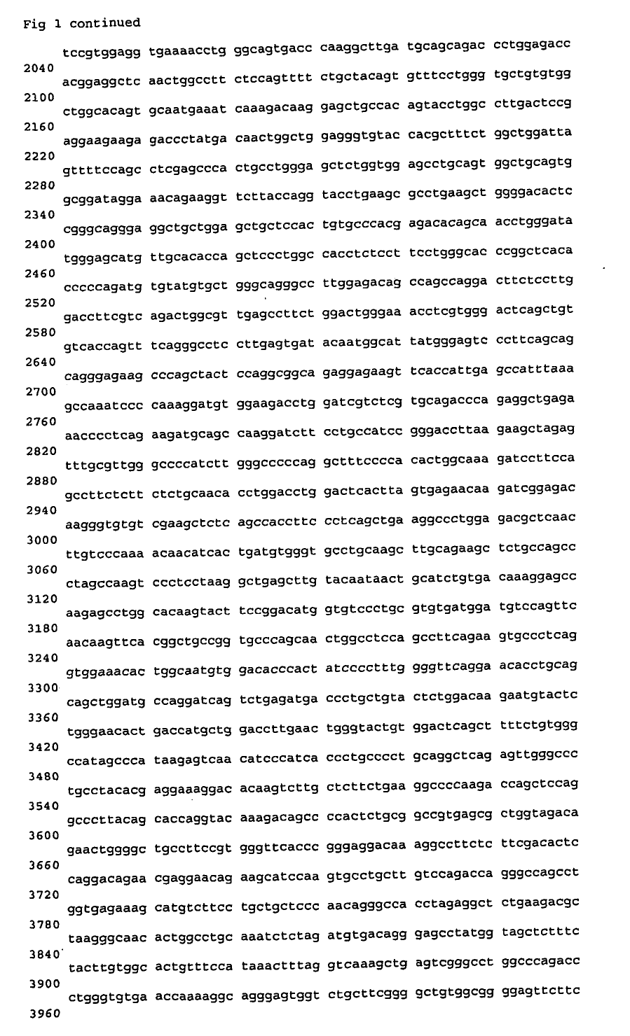

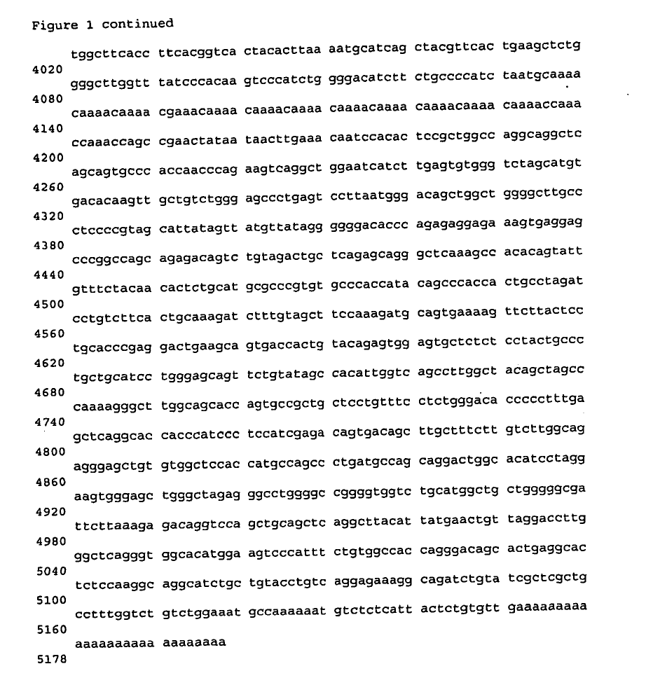

FIG. 1 is a DNA sequence comparison of mouse PTH receptor with modified signal sequence;

FIG. 2 shows the oligonucleotide sequences used to generate signal sequences;

FIG. 3 shows the oligonucleotide sequences used to produce CD4⁺ T-cell epitope;

FIG. 4 illustrates the main features of the expression vector according to the invention;

FIG. 5 is the nucleic acid sequence of CIITA; and

FIG. 6 is the nucleic acid sequence of PI31.

FIG. 7 is the nucleic acid sequence of HERNA helicase;

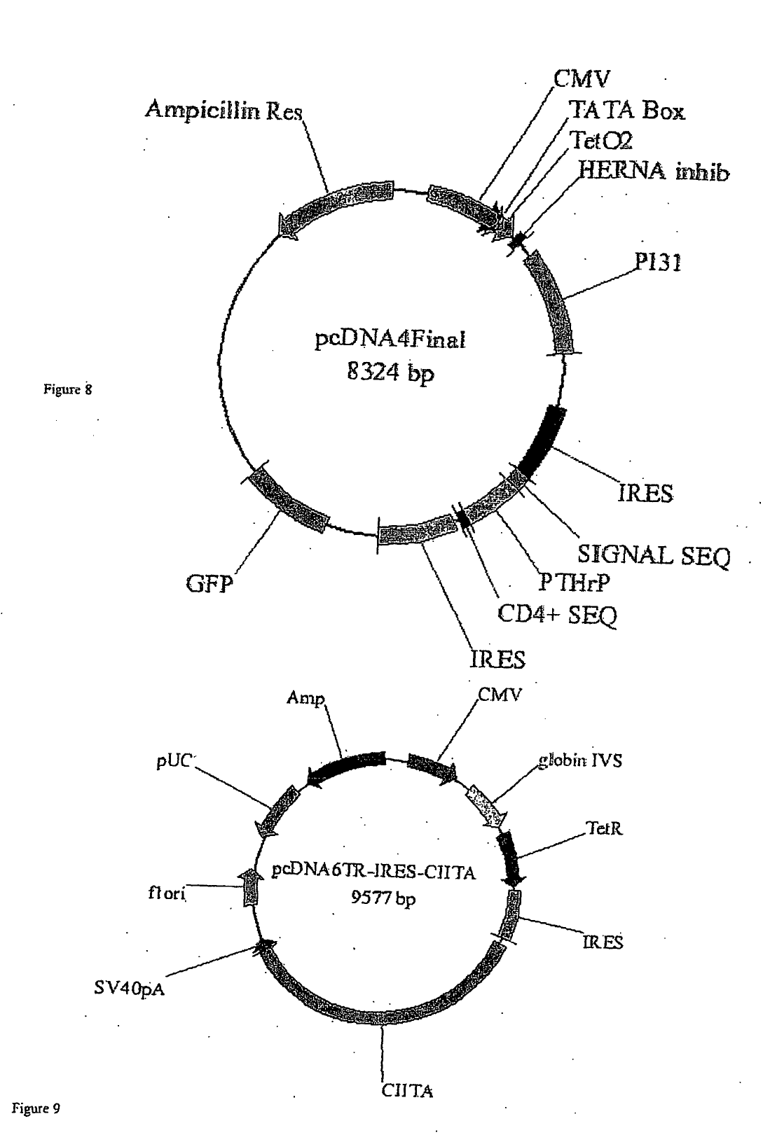

FIG. 8 is an example of a PI31 containing vector; and

FIG. 9 is an example of a CIITA containing vector.

### MATERIALS AND METHODS

The pIRES (Clontech) is the framework vector. A variety of tissue-specific promoter and enhancer sequences are used to target expression in a tissue specific manner. For this example the vector will be targeted to muscle via the murine muscle-specific creatinine kinase promoter which has been well characterised and is filed under GenBank accession number AF188002 which is incorporated by reference. The fragment (1355 bp) contains the MCK E1 enhancer and promoter and is sub-cloned into pBAD TOPO TA cloning vector (Invitrogen) using oligonucleotide primers specific for the forward and reverse flanking regions. The fragment is amplified by PCR using PfluTurbo (Stratagene) and 5′ extended oligonucleotide primers featuring 20 bp sequences which have homology to the region flanking the existing P_{CMVIE} promoter site within the pIRES vector. Integration/replacement of the P_{CMVIE} is achieved using the QuikChange XK site-Directed Mutagensis Kit (Stratagene) using the protocol described in Short Technical Reports (Biotechnqiues 31:88-92 July 2001).

The 816 bp CDS of the human PI31 gene (filed under GenBank accession number D88378 which is incorporated by reference) which is in a pcDNA3.1CTGFP vector is PCR amplified using PfuTurbo (Stratagene ) and 5′ extended oligonucleotides containing EcoR1 restriction sites spanning the flanking regions of the gene. Following agarose gel electrophoresis, excision, purification and EcoR1 treatment the PCR product is ligated into the linearised pIRES vector.

Sequences flanking the AUG translation initiation codon modulate the efficiency with which translation occurs. An optimal consensus of GCCG/ACCAUGG has been identified through extensive database mining analysis (Kozak 1987). The nucleotides in bold confer a strong context denoting a high likelihood of translation initation commencing from this codon. Divergence from these nucleotides results in a weak context, which can result in alternative translation initiation and leaky scanning.

Alternative translation initiation is the process by which translation commences at non AUG codons primarily CUG, ACG and GUG (Sun et al., 2001, Kevil et al., 1995). Combined with leaky scanning in which translation initiation occurs at the first AUG and other downstream initiation sites (Kozak 1990) alternative translation initiation results in the production in a population of ineffective truncated signal sequences. To address this the invention will contain a modified mouse signal sequence containing the optimal Kozak sequence, no CUG, ACG and GUG codons. To ensure correct cleavage the N-terminal codon of the cleavage boundary will be included, effectively adding one amino acid to the N-terminal of the translated protein. The signal sequence is based around the mouse parathyroid hormone receptor signal sequence and is designed to commence with an optimal Kozak sequence commencing 6 through to +4 relative to the ATG start codon. To prevent alternative translation initiation CTG codons are replaced with TTG, and GTG codons are replaced with GTA (see FIG. 1).

Instead of undertaking each of these modifications as mutagenesis procedures, forward and reverse oligonucleotides of 60 bp based on this modified sequence are purchased from Invitrogen and used in a low cycle number PCR using PfuTurbo (Stratagene) to generate a dsPCR product containing the sequence (see FIG. 2). Following agarose gel electrophoresis, band excision and purification, a second PCR is undertaken using the purified PCR product as template and a second set of forward and reverse oligonucleotides complimentary to the first 20 bp of the newly generated 5′ strands of the signal molecule and containing a 5′ 20 bp sequence with homology to the vector insertion region Following PCR, agarose gel electrophoresis, excision and purification this molecule is ligated into the 3′ IRES—5′ MCS (1700-1722) region of the vector using the QuikChange XK site-Directed Mutagenesis Kit.

This second MCS is used to clone in the GFP reporter molecule (719 bp) from pcDNA3.1CTGFP. As with PI31 5′ extended oligonucleotides containing Xba1 are used in PCR to facilitate ligation into the MCS linearised using the same enzyme.

A similar technique is used to produce the CD4+ T-cell epitope as was used for the signal sequence. The lymphocytic choriomeningitis virus (LCMV) contains an epitopic region spanning amino acids 61-80. Forward and reverse oligonucleotides are used in a low cycle number PCR using PfuTurbo (Stratagene) to generate a PCR product. A second PCR is undertaken using a second set of oligonucleotides containing 5′ 20 bp extensions homologous to the vector insertion site is undertaken. Ligation of the epitope is achieved using the QuikChange XK site-Directed Mutagenesis Kit.

The first 500 bp of the HERNA(helicase-MOI) CDS, is amplified by RT-PCR from mRNA extracted from HepG2 cells. However, to change its orientation upon mutagenesis, instead of the 5′ 20 bp extensions being homologous in the traditional forward orientation the extension for the forward oligonucleotide is the reverse oligonucleotide extension and visa versa so that upon integration the sequence will be incorporated into the vector in the reverse orientation. This fragment will replace the neomycin resistance gene downstream of the SV40Ori at position 3083.

Expression Evaluation

Transient transfections of differentiated skeletal muscle myoblasts (cell line C2C12) using calcium phosphate precipitation is undertaken using the vector constructs detailed in table 1. Quantitative expression of the reporter GFP gene is provided through FACS analysis.

The effectiveness of the signal sequence in directing the translated protein to the ER is determined by western blotting the conditioned media derived from experiment 4 using an anti-GFP monoclonal antibody (Clontech).

Immunological Evaluation

From the expression evaluation results, vectors are selected for immunological evaluation. The immunisation schedule is undertaken over a twelve week period using 6-8 week old BALB/c mice. Maxiprep (Sigma) purified plasmid DNA is resuspended in endotoxin free PBS at concentration of 5 mg/ml. During the immunisation period four sets of injections are given to the anaesthetised animal, intra muscularly at two sites, 200 l of pDNA per site in the flank of the right or left hind limb. Tail bleeds will be undertaken 7 days post injection.

To determine the titre of antisera Nunc maxisorp plates are coated with serial dilutions (1:10) of GFP starting from a stock at 2 g/ml in binding buffer and blocked with 5% skimmed milk powder. Diluted mouse antisera is added to the wells and incubated for 1 hour at room temperature. After washing goat anti mouse HrP secondary antisera (Dako) is added to the wells and incubated for 40 minutes at room temperature. Following a final wash step, TMB is added and allowed to develop for 30 minutes after which the reaction is stopped with concentrated sulphuric acid. The plate is read at 450 nm. To determine avidity, the highest dilution of GFP and mouse antisera consistent with an OD of approximately 1.5 are selected and used for an inhibition assay. Wells are coated with dilute GFP dilution, serial dilutions of soluble GFP are incubated with dilute mouse antisera for 1 hour at room temperature, following washing secondary antibody is added and the protocol completed as for the titration assay. The antisera demonstrating the greatest avidity will be those capable of detecting the least concentration of soluble GFP.

Vector Design RATTG

Starting with the sequences from a commercially available pair of tetracycline inducible mammalian vectors pcDNA4/TO and pcDNA6/TR, we have designed a pair of vectors cpcDNA4/TOHernaPI31ssPTHrPCD4+GFP and mupcDNA6/TR-IRES-CIITA(3) incorporating a number of features aimed at enhancing the secretion of the translated immunogen and bias DNA vaccination from an MHC class I towards an NHC class II event.

These designs have been accomplished using a combination of SimVector and Oligo6 software to map out each manipulation. The cpcDNA4/TOHernaPI31ssPTHrPCD4+GFP vector contains the majority of these features, which are described in detail in the sections below. The immunogen gene we have chosen for these initial experiments is PTHrP, however it is envisaged that this will be replaced with a multiple cloning site (MCS) in final versions of the vector enabling any gene to be ligated into the vector.

Parathyroid hormone-related protein (PTHrP) has been chosen, as we have previously expressed this protein in both bacteria and mammalian cells and have produced antibodies to specific regions which can be used during the characterisation and evaluation studies. The mupcDNA6/TR-IRES-CIITA(3) vector contains features for the inducibility and MHC class II expression.

Following DNA sequencing transfection of KCMH-1 and C3H cells and in vitro characterisation to determine the efficacy of HERNA inhibition and PI31 expression on the yield of GFP and PTHrP, DNA vaccination of mice is undertaken. Initially a series of reporter-induction protocols will be followed to study titration of tetracycline dosage and the kinetics of GFP reporter gene expression. Once optimal tetracycline dose has been established a series of duration-induction protocols is followed to study the effect of reducing the time of induction using full constructs (cpcDNA4/TOHernaPI31ssPTHrPCD4+GFP) and full constructs lacking PTHrP (cpcDNA4/TOHernaPI31GFP) on the magnitude of immune response using RIA and ELISA to determine titre and specificity. These results identify the minimum level of expression that can induce a humoral response. A series of time-induction protocols will then be followed during which the effects on the magnitude of immune response of a variety of induction timeframes (daily, weekly and fortnightly) will be tested using the same constructs. Again, antisera is evaluated by RIA and ELISA to determine anti-PTHrP specific titre and specificity. These results will identify optimal timeframes for induction. The results from these three induction studies will be used to develop a protocol that can be uniformly applied to all the constructs to confirm the efficacy of each feature or combination. Constructs will be targeted to the epidermis of skin (high turnover) and the skeletal muscle of the hindlimb (low turnover) using a commercial electroporator and gene gun respectively. Quantitative evaluations will be undertaken using PTHrP derived from PTHrP stably transfected human MCF-7, Hs578t breast tumour, and SaOS-2 osteosarcoma cell lines, in both a soluble and solid phase format.

All manipulations will be undertaken in a GMAG licensed laboratory to the highest standards of laboratory practice. cDNA for PI31, CIITA, IRES, PTHrP and GFP is available within the laboratory. The secretory and CD4+ sequences are purchased as oligonucleotides containing restriction endonuclease sites. cpcDNA4/TOHernaPI31ssPTHrPCD4+GFP are produced by producing PCR products for PI31, IRES, ssPTHrPCD4+ and GFP containing restriction endonuclease sites. Following digestion PI31 is ligated to IRES, PI31-IRES and ligated to ssPTHrPCD4+-IRES and P131-IRES-ssPTHrPCD4+-IRES are ligated to GFP. This cassette is introduced into the MCS of pcDNA4/TO to produce cpcDNA4/TOHernaPI31ssPTHrPCD4+GFP.

A number of site directed mutagenesis steps are required to remove restriction sites during this process. To remove the SV40 Zeomycin cassette a BstI1071 site has been identified upstream of the SV40pA, a similar site is introduced downstream of the SV40 ori by mutagenesis and the cassette removed following digestion. To incorporate the HERNA Helicase Inhibitory sequence an oligonucleotide will be purchased containing the antisense sequence flanked by a BspT1 restriction site. Digestion and ligation enables the sequence to be introduced between the CMV and PI31 sequences. The insertion of the IRES site between the genes will result in the production of single transcripts containing all the genes, enabling the order of gene translation to be controlled. MupcDNA6/TR-IRES-CIITA(3) will be produced by producing PCR products containing restriction sites for IRES and CIITA followed by digestion and ligation. Site Directed mutagenesis will be undertaken to remove and EcoR1 site within the TetR gene to a position downstream to enable the IRES-CIITA to be inserted.

TOP10 cells (Invitrogen) are transformed with constructs and grown as 250 ml cultures in Luria Broth. Plasmid DNA is extracted and purified using Qiagen HiSpeed Plasmid kits. Transfections of KCMH-1 and C3H cells will be accomplished using Effectene (Qiagen). For DNA vaccination plasmid DNA is introduced to keratinocytes of 6 week old in bred mice strains housed at the Biomedical Services Unit, University of Liverpool by electroporation. For each construct 20 mice will be immunised.

Modulation of Expression

Traditional immunisation occurs in a modulated manner through cycles of immunogen exposure, clearance and re-exposure. Vectors designed for use in producing antibodies require a mechanism to modulate gene expression thereby enabling progressively smaller quantities of the immunogen protein to be expressed as the immunisation programme progresses thereby leading to the selection of high affinity antibodies and enabling the IgM to IgG class shift to occur. The RATTG system is based upon the T-Rex™ system from Invitrogen which uses a two vector approach to facilitate gene expression on administration of tetracycline. Plasmid pcDNA6/TR is a regulatory vector that provides high levels of the tetracycline repressor (TetR) protein. Plasmid pcDNA4/TO contains the TetO2 site downstream of the TATA box of the CMV promoter. On co-transfection of pcDNA6/TR and pCDNA4/TO the TetR protein constitutively expressed by the former binds to the TetO2 site of the later and prevents transcription. Administration of excess tetracycline blocks the TetR binding site preventing it from binding to the TetO2 site and thus transcription proceeds. This system will enable expression to be modulated during the immunisation programme. Validation of induction will be evidenced through expression of a GFP gene. In vitro flow cytometry will be used to quantitate expression in the KCCMH-1 and C3H cells, in vivo a UV source will be used to identify GFP expression around the site of injection.

mRNA and Protein Degradation

During the early expression inductions, relatively high yields of the immunogen protein must be secreted from the cell in order to elicit a significant response. The vector must therefore incorporate features to prevent degradation of both mRNA and the translated protein.

Posttranscriptional gene silencing (PTGS) is a recently discovered phenomenon in which sequence specific mRNA degradation occurs following the introduction of transgenes into cells (Cogino et al., 2000). Small interfering RNA's of 21-25 nucleotides are generated from larger RNA strands, once produced these anneal to mRNA transcripts and target them for degradation by an as yet uncharacterized enzyme complex. A candidate molecule for both the cleavage of the siRNA precursor and the enzyme complex has been identified and is termed Dicer in Drosophila. (Moss 2001). Several studies in humans, flies and worms have demonstrated that Dicer (or species homolog) expression is required to produce siRNA's and facilitate PTGS (Grishok et al., 2001, Hutvagner et al., 2001, Knight et al., 2001). Down regulation of the recently cloned mammalian homolog A HERNA (helicase-MOI) accession number AB028449 (atsuda et al., 2000). We have designed an antisense HERNA sequence which we intend to incorporate into the pcDNA4/TO vector at position to inhibit the proteins expression and thereby potentially increasing the transcriptional efficiency of the vector.

Amongst a number of roles the proteasome a 700 kDa protease of 28 subunits is responsible for the degradation of cytoplasmic proteins, the peptide remnants of which become complexed with MHC class I complexes thereby inducing stimulation of CD8+ cytotoxic T cells. Inhibition of the proteasome has been demonstrated to block the degradation of most cytoplasmic proteins and the generation of MHC class I presented peptides. In order to minimise proteasomal degradation of the translated PTHrP we have incorporated a gene for the proteasomal inhibitor PI31 into the pcDNA4/TO vector at position.

Secretory Signal Sequence (sss)

Degradation of the translated PTHrP immunogen into peptides within the cytoplasm will result in MHC class I presentation. The pcDNA4/TO vector needs to incorporate a feature to direct cotranslation of the transcribed product into endoplasmic reticulum thereby minimising the presence of the immunogen protein within the cytoplasm. Sequestration of a translated protein within the cytoplasm occurs either when the mRNA transcript doesn't contain a signal sequence or when alternative translation initiation occurs when signal sequence is present giving rise to the production of a truncated ineffective signal sequence. Two features predispose a protein to alternative translation initiation, leaky scanning and alternative translation initiation codons. Leaky scanning occurs when a protein contains a suboptimal Kozak sequence which enables translation initiation to bypass the first ATG codon and commence initiation at either the next ATG codon or one of a number of alternatives. To address this a signal sequence based upon the mouse parathyroid hormone receptor signal sequence has been redesigned to commence with an optimal Kozak sequence commencing −6 through to +4 relative to the ATG start codon. To prevent alternative translation initiation within the sss. CTG codons have been replaced with TTG, and GTG codons with GTA. This sequence will be inserted upstream of PTHrP amino acid position 1

CIITA

A fundamental obstacle for using DNA vaccination is that despite incorporating proteasomal inhibitors and optimal signal sequences some of the translated immunogen protein will be sequestered to the cytoplasm where it be degraded by the proteasome and complexed with constitutively expressed MHC class I. To address this cells that the vector enters into are converted to antigen presenting cells through the over expression of MHC class II using MHC class II transactivator (CIITA). RFX5, RFXAP and CIITA are three recently cloned factors essential for the activation of MHC class II genes. Whilst the RFX factors are constitutively expressed CIITA is differentially expressed in a pattern that correlates with MHC class II genes, moreover it has recently been reported that CFTA quantitatively controls the level of MHC class II expression in mice. In this modification we introduce an IRES sequence downstream of the TR gene on the pcDNA6/TR vector downstream of which we insert the mouse CIITA gene (Accession number U60653). On co-vaccination with pcDNA4/TO the pcDNA6/TR vector will constitutively express the TetO2 repressor needed for the controlled induction and CIITA to upregulate MHC class II expression, consequently any sequestrated immunogen protein will be complexed with both MHC class I and class II.

CD4+ Th Epitope

When the primary sequence of the immunogen shares homology with that of an endogenous mouse protein, binding to the B cell receptor is believed to occur initially at regions of non-conservation. The whole molecule is internalised, digested and the peptides complexed with MHC class II on the surface of the cell. CD4+Th cells attempt to bind to the MHC class II-peptide complex through is receptors inducing the production of cytokines, which lead to clonal expansion of B cell. Binding is requires non conserved peptides. Therefore when an immunogen has no homology with endogenous proteins, its digestion will produce a large pool of peptides suitable for CD4+Th receptor binding. When only a small number exist the pool will reduce the opportunity for CD4+Th receptor binding

To ensure that on degradation the PTHrP immunogen peptide-MHC class II response binds to the CD4+Th receptor a sequence will be inserted downstream of PTHrP141 which encodes for a lymphocytic choriomeningitis virus (LCMV) CD4+ epitope.

Extraneous Viral Sequences

Both pcDNA4/TO and pcDNA6/TR contain two main sources of viral sequence the CMV promoter and the SV40 cassette. The CMV is replaced with a suitable non viral constitutive promoter amenable to induction through the Tetracycline system at the earliest opportunity. The SV40 cassette contains the Blasticin and Zeocin selectable antibiotic resistance genes neither of which are required, therefore both cassettes will be removed deleted from the vectors.

Vector Construction

PI31 -IRES-ssPTHrP1-141CD4+-IRES-GFP Insertion into pcDNA4/TO (Invitrogen)

IRES sequence EcoR1/Sal1 digested from the pIRES vector (Clontech)

PI31 proteasome inhibitor amplified by PCR from pooled Giant Cell Tumour cDNA using the following primers producing xbal-PI31_{—}96-1497

Fwd xbal Primer
CG TCT AGA TTT CCT CCA GAC GCC GTC
Rev Primer
GTG ATG TCA GGA GCA ATG GCA ATT A

xbal-PI31_{—}96-1497(cds127-942) digested with EcoR1(1235)to produce xbal-PI31-dEcoR1

d-denotes digested form

xbal-PI31-EcoR1 digested with xbal to produce dxbal-PI31-dEcoR1

dxbalPI31-dEcoR1 ligated with EcoR1 digested IRES Sal1 to produce dxbal-PI31-IRES-Sal1

PTHrP(1-141)amplified by PCR from an in house vector containing the gene using the following primers:

(i)General Sequence

Fwd Sal1 Primer
CCG TCG ACG ATG GAG CGG AGA CTG GTT C
Rev EcoR1 Primer
GGG AAT TCT GGG GGA GAC AGT TTT ATT CCA AT

(ii)Incorporation of Modified Signal Sequence

Fwd Sal1ss Primer
CC GTC GAC GCC ACC ATG GGG ACC GCC CGG ATC GCA CCC
AGC TTG GCG CTC CTT CTT TGC TGT CCA GTA CTC AGC
TCC GCA TAC GCG TTG GTA GCT GTG TCT GAA CAT CAG
CTC CTC CAT
Rev EcoR1 Primer
GGG AAT TCT GGG GGA GAC AGT TTT ATT CCA AT

(iii)Incorporation of CD4+T-Cell Epitope

Fwd Primer
CCG TCG ACG CCA CCA TGG GGA CCG CC
Rev CD4 EcoR1 Primer
GC GAA TTC TTA ATC AAA CTC CAC TGA TTT GAA CTG GTA
AAC GGG TTT ATA GAT GTA GGG ACC ATT AAG GCC ATG
CCT CCG TGA ATC GAG CTC CAG CGA CGT TGT

Sal1ssPTHrP1-141CD4+EcoR1 PCR product digested with Sal1/EcoR1 and ligated to dXbal-PI31-IRES-dSal1 to produce dSal1ssPTHrP(1-141)CD4dEcoR1

Site Directed Mutagenesis of dXbal-PI31-IRES-ssPTHrP1-141CD4+-dEcoR1 at EcoR1 1232 C/GAATTC AND Xbal 1868 TCTAC/GA

EcoR1/KspA1 digestion of IRES sequence from pIRES(Clontech)

GFP amplification by PCR from pcDNA3.1/CTGFP (Invitrogen) using the following primers to produce kspA1-GFP785-1767-xbal:

Fwd KspA1 Primer
CCG TTA ACG CGT GTA CGG TGG GAG GTC TAT
Rev Xba1 Primer
GGT CTA GAA ACA ACA GAT GGC TGG CAA CTA GAA

Digestion of KspA1-GFP785-1767-Xbal with KspA1 to produce dKspA1-GFP785-1767-Xbal

Site Directed Mutagenesis of Xbal 202 TCA/TAGA in dKspA1-GFP785-1767-kbal

Site Directed Mutagenesis of Xbal 637 TCA/TAGA in dEcoR1-IRES-dKspA11

Ligation of mudEcoR1-IRES-dKspA1 to mudKspA1-GFP785-1767-Xbal to produce mudEcoR1-IRES-GFP785-1767-Xbal

mu-denotes mutagenesis alteration

mudXbalPI31IRESssPTHrP1-141CD4dEcoR1 ligated to mudEcoR1-IRES-GFP785-1767-Xbal to produce mudXbalPI31IRE SssPTHrPCD4IRESGFPXbal

mudXbalPI31IRESssPTHrPCD4+IRESGFPXbal digested with Xbal and ligated into pcDNA4/TO

Removal of SV40Zeomycin Cassette

Site Directed Mutagenesis of Mls1 4710 TGGCCT/A and Bstl1071 4989 GTT/ATAC

Site Directed Mutagenesis at 6061 to introduce Bstl1071 site upstream of SV40ori

6064 GTGTGT mutated to GTATAC

Digestion of with Bstl1071 to Delete the SV40 Cassette

Incorporation of HERNA Helicase Inhibitory Sequence

Sequence Selected for antisenseRNA

| | |
|---|---|
| 5-3′ | CAATGAAAGA AACACTGGAT GAATGAAAAG |
| | CCCTGCTTTG CAACCCCTCA GCATGGCAGG |
| 3-5′ | cttactttct ttgtgaccta cttacttttc |
| | gggacgaaac gttggggagt cgtaccgtcc |
| 3-5′rev | cctgccgtgc tgaggggttg caaagcaggg |
| | cttttcattc atccagtgtt tctttcattc |

Incorporation of BspT1 Restriction Sites

Synthesized Template
GGCTTAAGCCTGCCGTGCTGAGGGGTTGCAAAGCAGGGCTTTTCATTCAT
CCAGTGTTTCTTTCATTCCTTAAGCGCAAAGAAAGTAAGGAATTCGC
Rev Primer
CGC TTA AGG AAT GAA AGA AAC

Double Stranded template produced using the rev oligo to prime the extension of the synthesized template using T4 DNA polymerase

Digestion of dsHERNA and mupcDNA4.with BspT1 and subsequent ligation of the sequence within the vector

IRES-CIITA Insertion into pcDNA6TR (Invitrogen)

IRES sequence EcoR1/xbal digested from the pIRES vector (Clontech)

CIITA amplified by PCR from mouse macrophage cDNA using the following primers producing xbal-CIITA-EcoR1

Forward xbal CIITA
CCT CTA GA GGG CAG CTG GAC TAC AGA CGT TAC T
Reverse EcoR1 CIITA
CGG AAT TC GCA GGG TGA TGG GAT GTT GAC TC
GCA GGG TGA TGG GAT GTT GAC TC

xbal-CIITA-EcoR1 digested with EcoR1 and xbal and ligated to produce

dXbalCIITA47-3457dEcoR1

Site Directed Mutagenesis of pcDNA6TR performed at 2324 to delete EcoR1 site and reinsertion it at 2340 (mutagenesis 2324 GAG/ATTC insertion 2340 GAATTC)

dEcoR1-IRES-CIITA-dEcoR1 ligated into mupcDNA6TR

Site Directed Mutagenesis at 7383 to insert a BstI1071 site between the flori and SV40 promoter

| | |
|---|---|
| 7381 | ATTAATTCTGTGGAATGTGTGTATAC |
| 7381 | ATGTATACTGTGGAATGTG |

SV40-blasticidin cassette deleted by digestion with BstI1071

1-58. (canceled)

59. A vector comprising a heterologous nucleic acid molecule encoding an antigenic polypeptide associated with a pathological condition and a further nucleic acid molecule selected from the group consisting of:
i) a nucleic acid molecule comprising the nucleic acid sequence of SEQ ID NO:2;
ii) a nucleic acid molecule which hybridizes under stringent conditions to the nucleic acid sequence of SEQ ID NO:2 and which encodes a protease inhibitor polypeptide; and
iii) nucleic acid molecules which comprise nucleic acid sequences which are degenerate because of the genetic code to the sequences in (i) and (ii) above.
wherein said vector is adapted for expression of polypeptides encoded by said heterologous nucleic acid molecule and by said further nucleic acid molecule.

60. A vector according to claim 59, wherein said vector is selected from the group consisting of plasmids, phagemids and viruses.

61. A vector according to claim 60, wherein said vector is a viral based vector based on a virus selected from the group consisting of adenoviruses, retroviruses, adeno-associated viruses, herpesviruses, lentiviruses, and baculoviruses.

62. A vector according to claim 59, wherein said heterologous nucleic acid molecule encodes an antigenic polypeptide derived from a viral pathogen.

63. A vector according to claim 62, wherein said viral pathogen is selected from the group consisting of Human Immunodeficiency Virus, Human T Cell Leukemia Virus (HTLV 1 & 2), Ebola virus, human papilloma virus (HPV), papovavirus, rhinovirus, poliovirus, herpesvirus, adenovirus, Epstein barr virus, and influenza virus.

64. A vector according to claim 59, wherein said heterologous nucleic acid molecule encodes an antigenic polypeptide derived from a bacterial pathogen.

65. A vector according to claim 64, wherein said bacterial pathogen is selected from the group consisting of Staphylococcus aureus, Staphylococcus epidermidis, Enterococcus faecalis, Mycobacterium tuberculsis, Streptococcus group B, Streptoccocus pneumoniae, Helicobacter pylor, Neisseria gonorrhea, Streptococcus group A, Borrelia burgdorferi, Coccidiodes immitis, Histoplasma sapsulatum, Neisseria meningitidis type B, Shigella flexneri, Escherichia coli, and Haemophilus influenzae.

66. A vector according to claim 59, wherein said heterologous nucleic acid molecule encodes an antigenic polypeptide derived from a parasitic pathogen.

67. A vector according to claim 66, wherein said parasitic pathogen is Trypanosoma Brucei spp or Plasmodium spp.

68. A vector according to claim 59, wherein said heterologous nucleic acid molecule encodes an antigenic polypeptide derived from a fungal pathogen.

69. A vector according to claim 68, wherein said fungal pathogen is Candida spp.

70. A vector according to claim 59, wherein said heterologous nucleic acid molecule encodes a tumor specific antigen.

71. A vector according to claim 70, wherein said antigen is selected from the group consisting of parathyroid hormone related protein, cathepsin K, and prostate specific antigen.

72. A vector according to claim 59, wherein said heterologous nucleic acid molecule is controlled by its cognate promoter.

73. A vector according to claim 59, wherein said heterologous nucleic acid molecule is controlled by a promoter which does not naturally control expression of the gene from which said heterologous nucleic acid molecule was derived.

74. A vector according to claim 73, wherein said promoter is a constitutive promoter.

75. A vector according to claim 74, wherein said promoter is derived from a gene selected from the group consisting of CMV promoter, SV40, chicken beta actin, telomerase reverse transcriptase, H⁺/K⁺ ATPase, and glyceraldehyde-3-phosphate dehydrogenase.

76. A vector according to claim 73, wherein said promoter is a regulatable promoter.

77. A vector according to claim 76, wherein said promoter is a cell or tissue specific promoter.

78. A vector according to claim 77, wherein said tissue specific promoter is derived from a gene selected from the group consisting of alkaline phosphatase; albumin; casein; prostate specific antigen; osteocalcin; cathepsin K; TRAP; RankL; PC8; cytokeratins 1,6,9,10,14,16; collagen type 1; elastin; NF-ATI (NF-Atp, NF-Atc2); tyrosinase; TRP-1, and muscle specific creatine kinase.

79. A vector according to claim 78, wherein said promoter is a muscle specific promoter.

80. A vector according to claim 79, wherein said muscle specific promoter is derived from a gene encoding MCK or myosin light chain 3F.

81. A vector according to claim 59, wherein said further nucleic acid molecule encodes a proteosome inhibitor.

82. A vector according to claim 81, wherein said inhibitor is mammalian PI31.

83. A vector according to claim 24 wherein said inhibitor is human PI31.

84. A vector according to claim 83, wherein expression of PI31 nucleic acid is controlled by its cognate promoter.

85. A vector according to claim 83, wherein the PI31 gene is controlled by a promoter which does not naturally control expression of the PI31 gene.

86. A vector according to claim 82, wherein the nucleic acid molecule encoding the PI31 is expressed co-ordinantly with said heterologous nucleic acid molecule.

87. A vector according to claim 59, wherein said vector comprises a nucleic acid molecule which encodes a polypeptide which stimulates expression of MHC class II; said nucleic acid molecule being selected from the group consisting of:
a) a nucleic acid molecule comprising the nucleic acid sequence of SEQ ID NO:1;
b) a nucleic acid molecule which hybridizes under stringent conditions to the nucleic acid of SEQ ID NO: 1 and which encodes a polypeptide which stimulates MHC class II expression; and
c) a nucleic acid molecules which comprise nucleic acid sequences which are degenerate because of the genetic code to the sequences in (a) and (b) above.

88. A vector according to claim 87, wherein said polypeptide is CIITA.

89. A vector according to claim 88, wherein said polypeptide is CIITA (DNA accession number U60653).

90. A vector according to claim 59, wherein said vector is further adapted to express an inhibitory RNA molecule, and wherein said inhibitory RNA is expressed from a DNA molecule selected from the group consisting of:
a) a DNA molecule comprising the polynucleotide sequence of SEQ ID NO:3;
b) a DNA molecule which hybridizes under stringent conditions to the polynucleotide sequence of SEQ ID NO:3 and which has helicase activity;
c) a DNA molecule which is degenerate because of the genetic code to those sequences in (a) and (b) above.

91. A method of inducing an immune response to an antigenic polypeptide associated with a pathological condition, said method comprising administering to an animal a vector according to claim 59.

92. A method according to claim 91, wherein said animal is a human.

93. A method according to claim 91, wherein said vector is administered by oral, intravenous, intraperitoneal, intramuscular, intracavity, subcutaneous, or transdermal injection.

94. An antibody which binds a polypeptide associated with a pathological condition obtainable by the method according to claim 91.

95. An antibody according to claim 94, wherein said antibody is a therapeutic antibody.

96. An antibody according to claim 94, wherein said antibody is a diagnostic antibody.

97. An antibody according to claim 96, wherein said diagnostic antibody is provided with a detectable label or tag.

98. An antibody according to claim 94, wherein said antibody is a monoclonal antibody or active binding fragment thereof.

99. An antibody according to claim 94, wherein said antibody is an opsonic antibody.

100. An antibody according to claim 98, wherein said antibody is a humanized antibody or a chimeric antibody.

101. A vector which is adapted for expression of a humanized or chimeric antibody according to claim 99.

102. A cell which has been transformed or transfected with a vector according to claim 101.

103. A method of producing a humanized or chimeric antibody comprising:
i) providing a cell transformed or transfected with a vector which comprises a nucleic acid molecule encoding a humanized or chimeric antibody according to claim 100;
ii) growing said cell in a growth environment conducive to production of said antibody; and
iii) purifying said antibody from said cell or said growth environment.

104. A hybridoma cell line which produces a monoclonal antibody according to claim 98.

105. A method of preparing a hybridoma cell-line which produces monoclonal antibodies obtained by administering a vector according to claim 59 to a host animal, said method comprising:
i) immunizing an immunocompetent mammal with the vector;
ii) fusing lymphocytes of the immunised immunocompetent mammal with myeloma cells to form hybridoma cells;
iii) screening monoclonal antibodies produced by the hybridoma cells of step (ii) for binding activity to the amino acid sequence encoded by the heterologous nucleic acid molecule comprised by said vector;
iv) culturing the hybridoma cells to proliferate and secrete the monoclonal antibody; and
v) recovering the monoclonal antibody from the culture supernatant.

106. A vaccine comprising a vector according to claim 59.

107. A vaccine according to claim 106, further comprising an adjuvant.

108. A method of vaccinating an animal against at least one pathological condition, said method comprising immunizing said animal with a vector according to claim 59.

109. A method according to claim 108, wherein said animal is a human.

110. A method according to claim 108, wherein said pathological condition is a viral infection.

111. A method according to claim 110, wherein said viral infection is selected from the group consisting of AIDS, herpes, rubeola, rubella, varicella, influenza, common cold, and viral meningitis.

112. A method according to claim 108, wherein said pathological condition is a bacterial infection.

113. A method according to claim 112, wherein said bacterial infection is selected from the group consisting of septicaemia, tuberculosis, bacterial food poisoning, blood infections, peritonitis, endocarditis, sepsis, bacterial meningitis, pneumonia, stomach ulcers, gonorrhoea, strep throat, streptococcal-associated toxic shock, necrotizing fasciitis, impetigo, histoplasmosis, Lyme disease, gastro-enteritis, dysentery, and shigellosis.

114. A method according to claim 108, wherein said pathological condition is a fungal infection.

115. A method according to claim 114, wherein said fungal infection is candidiasis.

116. A method according to claim 108, wherein said pathological condition is a parasitic infection.

117. A method according to claim 116, wherein said parasitic infection is selected from the group consisting of trypanosomiasis, malaria, schistosomiasis, and Chagas disease.


SEQUENCE LISTING (22 sequences)

SEQ ID NO: 1 - DNA, 5178 bp, Homo sapiens
acctgggcatctgaggactttttggagacttccggcacgccaggaggggcagctggactacagacgttactgcatcactctgctctctaaatcatgcgctgcctggttcctggcccttctgggtcttacctgccggagttgcaagaccatagtctgtgtgccaccatggatctgggatctccagagggcagctacctggaactccttaacagtgatgccgaccccctacatctctaccacctctatgaccagatggacctggctggggaggaggagatcgaactcagctcagagccagacacagataccatcaactgcgaccagttcagcaagctgttgcaggacatggaactggatgaagagacccgggaggcctatgccaacattgcggaactggatcagtacgtgttccaggatacccagctcgagggcctgagcaaggacctcttcatagagcacattggagcagaggaaggctttggtgagaacatagagatccctgtagaagcaggacagaagcctcagaagagacgcttcccggaagagcatgctatggactcaaagcacaggaagctagtgcccacctctaggacctcactgaactatttggatctccccactgggcacatccagatcttcaccactctgccccagggactctggcaaatctcaggggctggcacaggtctctccagtgtcctaatctaccacggtgagatgccccaggtcaaccaagtgctcccttcaagcagcctcagtatccccagtctccccgagtccccagaccggcctggttccaccagccccttcacaccatctgcagctgacctgcccagcatgcccgaacctgcgctgacctcccgtgtaaatgagacagaggacacatctccctccccatgccaagagggtcccgagtcttccatcaagcttccaaaatggccagaggctgtggagcgattccagcactccctacaggacaaatacaaggcattgccccagagcccaaggggtcctctggtggccgtggagctggtacgggccaggctggaaagaggcagcaacaagagccaggaaagggagctggccactcccgactggacagagcgccagctagcccacggtggtctggcagaggtacttcaggttgtcagtgactgcaggcgaccaggagagacacaggtggtcgctgtgctgggcaaggctggccagggaaagagccactgggccaggacagtgagtcacacctgggcatgtggccagttgctacaatatgactttgtcttctatgtcccctgtcattgcttggatcgtcccggggacacctaccacctgcgggatctgctctgtcccccgagcctgcagccactggccatggatgacgaggtccttgattatatcgtgaggcagccagaccgtgttctgctcatcctagatgctttcgaggagctagaggcccaagatggcctcctgcacggaccctgtggatctctgtccccagagccctgctccctccgaggactgctggctgggatcttccagcggaagctactgcgaggctgcacactgctcctcacagcccggccccggggccgcctggctcagagcctgagcaaggcagatgccatctttgaggtgcccagcttctctaccaagcaggccaagacttacatgaggcactactttgagaactcagggacagcggggaaccaagacaaggccctgggcctcctggagggccagcctcttctctgcagctatagtcacagccctgttgtgtgcagggctgtgtgccagctctccaaggccctgctagaacagggcacagaggcccagctaccttgtacacttacaggactctatgtcagcctgctaggtcctgcagctcagaacagtcctcccggagccttagtcgagctggccaagctggcctgggagctgggacgaagacaccaaagcaccttgcaagaaacccggttttcatccgtggaggtgaaaacctgggcagtgacccaaggcttgatgcagcagaccctggagaccacggaggctcaactggccttctccagttttctgctacagtgtttcctgggtgctgtgtggctggcacagtgcaatgaaatcaaagacaaggagctgccacagtacctggccttgactccgaggaagaagagaccctatgacaactggctggagggtgtaccacgctttctggctggattagttttccagcctcgagcccactgcctgggagctctggtggagcctgcagtggctgcagtggcggataggaaacagaaggttcttaccaggtacctgaagcgcctgaagctggggacactccgggcagggaggctgctggagctgctccactgtgcccacgagacacagcaacctgggatatgggagcatgttgcacaccagctccctggccacctctccttcctgggcacccggctcacacccccagatgtgtatgtgctgggcagggccttggagacagccagccaggacttctccttggaccttcgtcagactggcgttgagccttctggactgggaaacctcgtgggactcagctgtgtcaccagtttcagggcctccttgagtgatacaatggcattatgggagtcccttcagcagcagggagaagcccagctactccaggcggcagaggagaagttcaccattgagccatttaaagccaaatccccaaaggatgtggaagacctggatcgtctcgtgcagacccagaggctgagaaacccctcagaagatgcagccaaggatcttcctgccatccgggaccttaagaagctagagtttgcgttgggccccatcttgggcccccaggctttccccacactggcaaagatccttccagccttctcttctctgcaacacctggacctggactcacttagtgagaacaagatcggagacaagggtgtgtcgaagctctcagccaccttccctcagctgaaggccctggagacgctcaacttgtcccaaaacaacatcactgatgtgggtgcctgcaagcttgcagaagctctgccagccctagccaagtccctcctaaggctgagcttgtacaataactgcatctgtgacaaaggagccaagagcctggcacaagtacttccggacatggtgtccctgcgtgtgatggatgtccagttcaacaagttcacggctgccggtgcccagcaactggcctccagccttcagaagtgccctcaggtggaaacactggcaatgtggacacccactatcccctttggggttcaggaacacctgcagcagctggatgccaggatcagtctgagatgaccctgctgtactctggacaagaatgtactctgggaacactgaccatgctggaccttgaactgggtactgtggactcagcttttctgtgggccatagcccataagagtcaacatcccatcaccctgcccctgcaggctcagagttgggccctgcctacacgaggaaaggacacaagtcttgctcttctgaaggccccaagaccagctccaggcccttacagcaccaggtacaaagacagccccactctgcggccgtgagcgctggtagacagaactggggctgccttccgtgggttcacccgggaggacaaaggccttctcttcgacactccaggacagaacgaggaacagaagcatccaagtgcctgcttgtccagaccagggccagcctggtgagaaagcatgtcttcctgctgctcccaacagggccacctagaggctctgaagacgctaagggcaacactggcctgcaaatctctagatgtgacagggagcctatggtagctctttctacttgtggcactgtttccataaactttaggtcaaagctgagtcgggcctggcccagaccctgggtgtgaaccaaaaggcagggagtggtctgcttcggggctgtggcggggagttcttctggcttcaccttcacggtcactacacttaaaatgcatcagctacgttcactgaagctctggggcttggtttatcccacaagtcccatctggggacatcttctgccccatctaatgcaaaacaaaacaaaacgaaacaaaacaaaacaaaacaaaacaaaacaaaacaaaacaaaaccaaaccaaaccagccgaactataataacttgaaacaatccacactccgctggccaggcaggctcagcagtgcccaccaacccagaagtcaggctggaatcatcttgagtgtgggtctagcatgtgacacaagttgctgtctgggagccctgagtccttaatgggacagctggctggggcttgccctccccgtagcattatagttatgttataggggggacacccagagaggagaaagtgaggagcccggccagcagagacagtctgtagactgctcagagcagggctcaaagccacacagtattgtttctacaacactctgcatgcgcccgtgtgcccaccatacagcccaccactgcctagatcctgtcttcactgcaaagatctttgtagcttccaaagatgcagtgaaaagttcttactcctgcacccgaggactgaagcagtgaccactgtacagagtggagtgctctctcctactgccctgctgcatcctgggagcagttctgtatagccacattggtcagccttggctacagctagcccaaaagggcttggcagcaccagtgccgctgctcctgtttcctctgggacaccccctttgagctcaggcaccacccatccctccatcgagacagtgacagcttgctttcttgtcttggcagagggagctgtgtggctccaccatgccagccctgatgccagcaggactggcacatcctaggaagtgggagctgggctagagggcctggggccggggtggtctgcatggctgctgggggcgattcttaaagagacaggtccagctgcagctcaggcttacattatgaactgttaggaccttgggctcagggtggcacatggaagtcccatttctgtggccaccagggacagcactgaggcactctccaaggcaggcatctgctgtacctgtcaggagaaaggcagatctgtatcgctcgctgcctttggtctgtctggaaatgccaaaaaatgtctctcattactctgtgttgaaaaaaaaaaaaaaaaaaaaaaaaaaa

SEQ ID NO: 2 - DNA, 3188 bp, Homo sapiens
  misc_feature (1099)..(1099): n is a, c, g, or t
attcgcggccgctgcaagaaccagcgcaagagggaagcggagttatagctaccccggccgcggagccggctcactgcactacccccgcccccttctttcctccagacgccgaagtcgcgggcgctcatggcgggcctggaggtactgttcgcatcggcagcgccggccatcacctgcaggcaggacgcgctcgtctgcttcttgcattgggaagtggtgacacacggttactgcggcttgggtgtcggtgaccagccgggtcccaatgataagaagtcagaactgctgccagctgggtggaacaacaataaagacctgtatgtcctccggtatgagtataaggatgggtccagaaagctccttgtgaaagccatcaccgtggagagcagcatgatcctcaatgtgctggaatatggctcacagcaagtggcagacttgaccctgaacttggatgattatattgatgcagaacacctgggtgacttccacaggacctacaagaacagtgaggagcttcggtctcgtattgtgtctggaatcatcacacctatccatgagcagtgggaaaaggctaatgtaagcagtccccaccgggagttcccccctgctaccgccagagaggtggacccactccggattcctccacaccacccacacaccagtcggcagcctccctggtgtgatcccctgggcccgtttgttgtcgggggagaagacttagacccttttgggcctcggagaggtggcatgattgtggatcccctgagatctggcttcccaagagcacttattgacccttcctcaggcctcccgaaccgacttcctccaggcgctgtgcccccaggagctcgctttgacccctttggacccattgggaccagcccacccggacctaacccagaccatctccccccgccgggctacgatgacatgtacctgtgaaggcctcaagaatgtaacatcccaggcttccctccattctcctggagctgccaccgctgtccccatcagcaaccatgttcttgcaggctgggggcaagggattctgctcatgtgtgtggagaccggctgggatagcctccccaccccttatcagagncaagacacctgctggagctctccacctagctggagatagctcccaaagagaaatcagtgtgtctcttncaccatcagctcctccccttacaccaccagctcctctccacttcccangggagactccggcanccttcagcaacatatatcctcgaccagatgcagtgctataagaacagaacgcattttggatgttattattaagaaccaaatgtcaatacagaattcatgttgccggtttcccacttttctttttacattaatgcatagctgcttccatttatgagactttagagtttgagtttctgtagggctgaatgactctttttcctgcccagggcccattcttgcttctcaggcaccttccgtttattaattgccattgctcctgacatcactaagatgggtccccttctggctgcatgaatggaaatgagtgactggaaatcccataggccacaagaatgactttcacaagggcaggaacattgtggaaagactgcatcattctgatgaggcaaaatcctccagctattcctgtctgggccagttttgtaggtccatctgtgcatgggcagcagtagtcacaaagccaagganaaaacagagcagacctgaaggctaatcttatttttgccactaacttagtgantgaccctaagcaagttccttctcctcttagggccttgtgccaagcctatgaaattggaggtgnctttcctgctctaaagcattttgatgtctcattctgtgtttggtaacccctataaactggggcagaggaaaagaatgatggttcaaggccatacttcccttgaaccttgtgtggttcttgcctaactctgtggtttttggaccccatggggcccagacagagcacaggagcatgggctgcctctgagtgtggtgttgaacttcgggaggagcagggagccctgcaccttgtgtcctggcccacctgacctttggtgttctccggatccttttcagcccgaggcctgacagacgcgggcagtgatgagccctgttctggagtggaaagagcacgatagagcaccaggctaagaggcacgagatcaaggcggtagtcacttccgctctgcagctagcatttcaaccatatgtggatcctttcatttctcagctccctggattccttcccctaaattaggacctattatttacctgtaggtaagcaagctactgtagctcttctgaggtatctcccaggctgttttctgtagcctcagantgcctatctncttagcctgagaacaggtagatgnaaactaaactgatgcctaggcccagggtcagtctcagatggaagctgggcctgggtggggaggctagcatgcgtggctccctgggtatttctgtcagtccccatggcaagcagtgatttagtaaaacaccccagagtcagggaagccaaccaccttgaaacctttaggacatctctgctttggagaaagacccagagatcaggcagaggtgcagattcantcattactcataacctttgagagatgtcacntgggnggagtgttagtctttgttttggagntgggccattcttgcaccccccaggacttagagcagtttgntcataaagacatcctttattataaaaggaagtatttataggatgatagagaccatcagatagaagcagggggggtagataacttttaggnccttgatgtgtggagaagataaaatttaaacaataaatttgccacttagattttctancaccacagtccgacgaaagatagttatatacaacattctgttttctgataacaactgtgattcaccttcagaattggccattttttttgtgagtttccttgcatcaaggacactgagaaacacagtcattgtcttaggtgttctatgggaggaagtgaatagagcctttaggaacttcctggtcaagcttatggtgcttattttgatctgggccacttccctccttccagtcatgagtaatcatcaaggagcaagttggagtgtttcaggtgtatattttgtagaacccaaaagattggagccttaacaataaacatcagagcggccgcgaattc

SEQ ID NO: 3 - DNA, 240 bp, Homo sapiens
tctcaaacattaaaacgtaagctgtgctagaacaaaaatgcaatgaaagaaacactggatgaatgaaaagccctgctttgcaacccctcagcatggcaggcctgcagctcatgacccctgcttcctcaccaatgggtcctttctttggactgccatggcaacaagaagcaattcatgataacatttatacgccaagaaaatatcaggttgaactgcttgaagcagctctggatcataata

SEQ ID NO: 4 - DNA, 26 bp, Homo sapiens
cgtctagatttcctccagacgccgtc

SEQ ID NO: 5 - DNA, 25 bp, Homo sapiens
gtgatgtcaggagcaatggcaatta

SEQ ID NO: 6 - DNA, 28 bp, Homo sapiens
ccgtcgacgatgcagcggagactggttc

SEQ ID NO: 7 - DNA, 27 bp, Homo sapiens
gggaattctgggggagacagttttatt

SEQ ID NO: 8 - DNA, 119 bp, Homo sapiens
ccgtcgacgccaccatggggaccgcccggatcgcacccagcttggcgctccttctttgctgtccagtactcagctccgcatacgcgttggtagctgtgtctgaacatcagctcctccat

SEQ ID NO: 9 - DNA, 32 bp, Homo sapiens
gggaattctgggggagacagttttattccaat

SEQ ID NO: 10 - DNA, 26 bp, Homo sapiens
ccgtcgacgccaccatggggaccgcc

SEQ ID NO: 11 - DNA, 104 bp, Homo sapiens
gcgaattcttaatcaaactccactgatttgaactggtaaacgggtttatagatgtagggaccattaaggccatgcctccgtgaatcgagctccagcgacgttgt

SEQ ID NO: 12 - DNA, 30 bp, Homo sapiens
ccgttaacgcgtgtacggtgggaggtctat

SEQ ID NO: 13 - DNA, 33 bp, Homo sapiens
ggtctagaaacaacagatggctggcaactagaa

SEQ ID NO: 14 - DNA, 60 bp, Homo sapiens
caatgaaagaaacactggatgaatgaaaagccctgctttgcaacccctcagcatggcagg

SEQ ID NO: 15 - DNA, 60 bp, Homo sapiens
cttactttctttgtgacctacttacttttcgggacgaaacgttggggagtcgtaccgtcc

SEQ ID NO: 16 - DNA, 60 bp, Homo sapiens
cctgccgtgctgaggggttgcaaagcagggcttttcattcatccagtgtttctttcattc

SEQ ID NO: 17 - DNA, 97 bp, Homo sapiens
ggcttaagcctgccgtgctgaggggttgcaaagcagggcttttcattcatccagtgtttctttcattccttaagcgcaaagaaagtaaggaattcgc

SEQ ID NO: 18 - DNA, 21 bp, Homo sapiens
cgcttaaggaatgaaagaaac

SEQ ID NO: 19 - DNA, 33 bp, Homo sapiens
cctctagagggcagctggactacagacgttact

SEQ ID NO: 20 - DNA, 54 bp, Homo sapiens
cggaattcgcagggtgatgggatgttgactcgcagggtgatgggatgttgactc

SEQ ID NO: 21 - DNA, 26 bp, Artificial
  oligonucleotide primer
attaattctgtggaatgtgtgtatac

SEQ ID NO: 22 - DNA, 19 bp, Artificial
  oligonucleotide primer
atgtatactgtggaatgtg